(19) United States
(12) Patent Application Publication
(10) Pub. No.: US 2025/0382570 A1
(43) Pub. Date: Dec. 18, 2025

(54) GENERATION AND APPLICATION OF FUNCTIONAL HUMAN ASTROCYTES FROM PLURIPOTENT STEM CELLS

(71) Applicant: The Regents of the University of California, Oakland, CA (US)

(72) Inventors: Edsel M. ABUD, Escondido, CA (US); Wayne W. POON, Anaheim, CA (US); Ricardo RAMIREZ, Hyde Park, MA (US)

(21) Appl. No.: 18/880,063

(22) PCT Filed: Jun. 30, 2023
(86) PCT No.: PCT/US2023/026799

§ 371 Date: Dec. 30, 2024

Related U.S. Application Data
(60) Provisional application No. 63/367,579, filed on Jul. 1, 2022.

(51) Int. Cl. C12N 5/0793; A61K 35/30

(52) U.S. Cl. CPC C12N 5/0619; A61K 35/30; C12N 2506/08; C12N 2506/45; C12N 2533/52

(57) ABSTRACT

A method of producing human astrocytes from neural stem cells (NSCs) involves providing induced progenitor cells (iPSCs), differentiating iPSCs to neural stem cells (NSCs), and differentiating NSCs to astrocytes.

### CROSS-REFERENCE TO RELATED APPLICATION

This application claims priority under 35 U.S.C. §119(e) to U.S. Provisional Application No. 63/367,579, filed Jul. 1, 2022, the contents of which are incorporated herein by reference in their entireties.

### STATEMENT OF GOVERNMENT SUPPORT

This invention was made with government support under Grant No. AG016573 and Grant No. P30AG066519 awarded by the National Institutes of Health (NIH). The government has certain rights in the invention.

### BACKGROUND

The present disclosure is related to differentiation and generation of human astrocytes from pluripotent stem cells that are useful in various applications, including the study of astrocyte biology and applications of the generated astrocytes. Astrocytes are the most abundant central nervous system cell type and have been implicated in the pathobiology of many neurological diseases. Thus, a need in the art exists to prepare human astrocytes from pluripotent stem cells. However, prior methods have failed, as described below.

### PRIOR ART IPSC-BASED SMALL MOLECULE METHODS

Krencik et al., Specification of transplantable Astroglial Subtypes from Human Pluripotent Stem Cells, Nat Biotechnol. 29(6): 528-534 (2011) and Krencik et al., Dysregulation of astrocyte extracellular signaling in Costello syndrome, Sci Transl. Med. 6;7 (286) (2015) described the generation of immature astrocytes over the course of >120 days that proceeds through an EB-based neuroepithelial precursor stage producing immature astroglia with dorsal telencephalic characteristics. Regio-specific astrocytes can be achieved by adding retinoic acid or sonic hedgehog to caudalize (HOXB4 vs OTX2) or ventralize (NKX2-1) progenitors (day 10-21) prior to astrocyte differentiation with EGF and FGF2 (day 21-90). At day 90, the resulting cells resemble immature astrocytes in which 10% are GFAP positive and after removal of growth factors, transition to >90% of cells expressing S100b and GFAP (day 120-180) and only express the mature astrocyte marker, aquaporin-4 (AQP4), after day 210. This prior art protocol is time intensive taking (over 6 months) with homogenous astrocytes only beginning to express AQP4. Also, while key astrocyte markers are included, an analysis on the whole transcriptome level is not carried out to properly validate the identity of iPS-derived astrocytes in comparison to primary sources. Disadvantages: Very long time period to generate astrocytes making it difficult to generate cells in a timely manner for studies or therapies and may be immature astrocytes (See Krencik et al, Human astrocytes are distinct contributors to the complexity of synaptic function, Brain Re. Bull 129:66-73 (2017) and Krencik et al., Systematic Three-Dimensional Coculture Rapidly Recapitulates Interactions between Human Neurons and Astrocytes, Stem Cell Reports 9(6): 1745-1753 (2017).

Serio et al., Astrocyte pathology and the absence of non-cell autonomy in an induced pluripotent stem cell model of TDP-43 proteinopathy, Proc. Nat'l Acad. Sci. USA 110(12) 4697-702 (2013) described the generation of functional astrocytes derived from a neural precursor cell (NPC). First, they generate NPCs as cultured neurospheres in EGF/LIF-containing media (28-42 days) to enrich for astroglial progenitors. These Vim+/NFIA+ precursors can be expanded with EGF and FGF2 but less than 30% express GFAP. Subsequent differentiation with CNTF (days 42-56) yielded >90% GFAP. iPSC-derived astrocytes exhibit glutamate uptake, promote synaptogenesis, and propagate calcium waves. Similarly, whole transcriptome analysis is not carried out to validate the identity of iPS-derived astrocytes and instead only focuses on a subset of astrocyte markers that do not include ALDH1L1 and AQP4, a mature astrocyte marker. This method has disadvantages such as failure to establish maturation and incomplete functional characterization.

Juopperi et al., Astrocytes generated from patient induced pluripotent stem cells recapitulate features of Huntington's disease patient cells, Mol. Brain 5:17 (2012) described the generation of astrocytes from neurospheres dissociated into single cells, plated onto poly-L-ornithine-laminin, and uses a commercial media source (ScienCell). Again, the astrocytes are poorly characterized at the transcriptome level. Furthermore, while the components of the commercial media source are proprietary, ScienCell requires addition of Fetal Bovine Serum (FBS) to complete their media. Astrocytes developed with FBS can artificially and unreliable pseudoenrich for astroglial markers, like GFAP. Furthermore, the immunocytochemical analysis reveals that the astrocytes appear reactive when compared to astrocytes isolated in serum-free media ex vivo. A reactive astrocyte phenotype is not conducive to study and elucidate neurodegenerative mechanisms that may result from astrogliosis. Reactive astrocytes are not conducive to multi-culture with other CNS cells, such as neurons and microglia. Disadvantages: Use of serum which activates astrocytes making it difficult to use cells to examine aspects of healthy function and neuroinflammation.

Roybon et al., Human stem cell-derived spinal cord astrocytes with defined mature or reactive phenotypes, Cell Rep. 4(5):1035-1048 (2013) utilized a dual SMAD inhibitor strategy (SB431542 inhibits ALK4/5/7 and LDN193189 inhibits TGFβ1) to neuralize human stem cells grown on MEFs to neural progenitors (PAX6+/Oct4−) and included RA and SHH-C to yield caudal ventral specification to produce spinal cord astrocytes in differentiation media that includes BDNF, GDNF, IGF, and CNTF in a serum-containing media formulation. Astrocytes were not compared to primary spinal astrocytes at the whole transcriptome level but relied on the presence or absence of OTX2 and HOXB4, factors to assess specification. To study many neurological diseases requires brain-specific astrocytes. Additionally, serum is known to activate astrocytes and therefore, studying astrogliosis is challenging when the cells are already activated. Thus, spinal cord astrocytes may not fully recapitulate the phenotype of brain astrocytes. Disadvantages: Generation of spinal cord astrocytes without comparison. Use of serum which activates astrocytes making it difficult to use cells to examine aspects of neuroinflammation and is not reproducible.

Shaltouki et al., Efficient generation of astrocytes from human pluripotent stem cells in defined conditions, Stem Cells (5):941-52 (2013) described astrocytes differentiated after 35 days from neural stem cells (NSC) NSCs using CNTF, BMP2, heregulin, IGF1, activing, and FGF2 in a DMEM/F12-based media. Astrocyte genomic indentity was evaluated using microarray analysis, which revealed that the differentiated astrocytes cluster separately from NSCs as astrocytes are differentiated. While this is the first paper to describe a transcriptome analysis, it is somewhat limited due to the high rate of false positives with the microarray platform versus RNAseq analysis, which more accurately assesses gene expression. Also, a comparison to primary astrocytes was not included which further limits the interpretation of their gene expression analysis. Disadvantages: Many investigators highlight the lack of reproducibility of this method.

Tcw et al., An Efficient Platform for Astrocyte Differentiation from Human Induced Pluripotent Stem Cells, Stem Cell Reports 9(2):600-614 (2017) described the generation of astrocytes from NPCs, following NPC differentiation, the NPCs were exposed to ScienCell commercial differentiation media that contains serum. This protocol showed that astrocytes can be generated from multiple iPSC lines. There are several limitations to this method: 1) The presence of serum in the media is an issue as it has been established that serum activates astrocytes and exposes astrocytes to factors not normally seen in the brain under non-pathological conditions. 2) The media formulation is unknown, and users of this protocol are dependent on unknown factors when designing studies to examine factors impacting astrocyte biology 3) Morphologically, while a number of early astrocyte markers are expressed, late astrocyte markers are not expressed, which could suggest that the generated cells are actually radial glia or glia progenitors, as the cells do not exhibit the typical astrocyte morphology. Disadvantage: Use of commercial and proprietary serum-based media. The use of proprietary media prevents appropriate troubleshooting when generating cells and limits functional data interpretation due to non-disclosure of factors that can influence geno-and phenotype. Serum source is not disclosed, and serum is well known to be a variable product in cell culture medium. Serum use also leads to activated astrocyte phenotype making it difficult to use cells to examine aspects of neuroinflammation and more recently, the transcriptome data highlights that they are in fact astrocyte progenitors that require maturation in vivo with other brain cells (Preman et al., Human iPSC-derived astrocytes transplanted into the mouse brain undergo morphological changes in response to amyloid-β plaques, Molecular Neurodegeneration 16, 68 (2021)).

### Transcription Factor-Based Approaches

Canals et al., Rapid and efficient induction of functional astrocytes from human pluripotent stem cells, Nat. Methods 15(9):693-696 (2018) used NFIB and SOX9 to induce astrocytes from iPSCs using viral transduction. The major disadvantage of this protocol is the need for serum starting at 10% that is eventually lowered to 1%. The cells morphologically resemble primary astrocytes. Functionally, ICC was used to examine protein expression and they also exhibited the capability to store glycogen, a key functional feature, qPCR of key astrocyte canonical genes, facilitate neuron network activity when co-cultured with iNs, display calcium transients.

Tchieu et al., NFIA is a gliogenic switch enabling rapid derivation of functional human astrocytes from pluripotent stem cells, Nature Biotechnology 37, 267-275 (2019) used NFIA to trigger a gliogenic switch in astrocytes at a NSC stage leading to a more rapid astrocyte generation than traditional methods. The RNAseq analysis reveals similarity to fetal astrocytes. Functionally, they can enhance synaptogenesis, facilitate calcium transients, can adopt the A1 phenotype, and incorporate into the brain of murine models. The NFIA transiently alters chromatin accessibility of astrocyte gene promoter regions to make them competent for astrocyte commitment following addition of LIF. In the absence of LIF, the cells revert to NSCs with doxycycline removal, suggesting the NFIA switch is transient. Importantly, the astrocytes demonstrate increased maturation when co-cultured with neurons, suggesting they are not mature without additional neuronal factors.

Major disadvantages of Tx factor approaches include: while the time to generate astrocytes is accelerated, the ability to cryopreserve cells is poor, suggesting some aspect of transcription factor expression alters cell viability in the cryopreservation/thaw process. Additionally, the Canals method uses serum and Tchieu still require neuronal maturation.

### Organoid Approaches

Sloan et al., Human Astrocyte Maturation Captured in 3D Cerebral Cortical Spheroids Derived from Pluripotent Stem Cells, Neuron 95(4):779-790 (2017) used an organoid 3D-based approach to generate mature astrocytes that requires greater >1 year. However, the transcriptome is similar to ex vivo human astrocytes again highlighting the need for neurons for providing an environment to generate relevant astrocyte phenotypes. RNAseq analysis identifies expression of adult human astrocyte markers (Zhang et al., Purification and Characterization of Progenitor and Mature Human Astrocytes Reveals Transcriptional and Functional Differences with Mouse, Neuron 89(1): 37-53 (2016)) and ICC of d295 astrocytes reveal that they exhibit morphologies similar to human primary astrocytes. The astrocytes promote neuron synaptic maturation and electrophysiological properties.

Krencik et al., Systematic Three-Dimensional Coculture Rapidly Recapitulates Interactions between Human Neurons and Astrocytes, Stem Cell Reports 9(6): 1745-1753 (2017) used 3D assembloids to combine neurons and astrocytes to achieve a more relevant in vitro brain model in which the astrocytes exhibit morphologies similar to primary human astrocytes from astrocytes derived in Krencik et al., 2011 studies. Therefore, this method requires providing cues from iNs to generate a more relevant astrocyte phenotype. While mouse astrocytes exhibit tiling, human astrocytes do not. This was exhibited in vitro and is one of many differences between mouse and human astrocytes.

Barbar et al., CD49f Is a Novel Marker of Functional Reactive Human iPSC-Derived Astrocytes, Neuron 107(3):436-453 (2020) used neural induction followed by organoid generation and then subsequent isolation of CD49f positive astrocytes. The astrocytes exhibit calcium transients, uptake glutamate, exhibit neuronal support (synaptic numbers and electrophysiology) and phagocytosis, and can adopt an Al-like phenotype. Although morphology looks like primary astrocytes, many functional endpoints were NOT compared to primary cells. By RNAseq, they cluster with TCW astrocytes and by scRNAseq, they are still not 100% pure.

There are many disadvantages to these methods, for example, scale up requires an extended time in culture i.e., almost one year for Sloan method and an additional period of time for Krencik which is in addition to the initial time for the method described above for the small molecule derivation of astroctyes requiring >200 days.

Despite numerous methods to generate astrocytes there are still many limitations and disadvantages of the prior art as follows: lack of reproducibility across multiple healthy and disease lines; use of serum which activates the cells limiting utility; maturation of cells from a radial glia or astrocyte progenitor state to mature astrocytes require additional co-culture; extended times needed in culture to achieve an astrocyte; comparison to primary cells is incomplete including functional comparisons.

### SUMMARY OF THE DISCLOSURE

A method of producing human astrocytes from neural stem cells (NSCs) is provided. In another aspect, this disclosure provides a method for generating human astrocytes from human neural stem cells produced from human induced pluripotent stem cells (iPSCs). Applicant has found that the cells produced by these methods produce human astrocytes that function as mature neurons, as assessed by electrophysiology. The human astrocytes produced by the method of this disclosure have promote blood brain barrier (BBB) function, can engraft in brain, uptake glutamate, and phagocytose.

In one aspect, a method of producing neural stem cells (NSCs) from human induced progenitor cells (iPSCs), is provided, the method comprising, or alternatively consisting essentially of, or yet further consisting of, culturing the iPSCs in completeTeSR-E8 culture medium in the absence of feeder cells for an effective amount of time, thereby producing human neural stem cells from human iPSCs. In one embodiment, the culturing step comprises, or consists essentially of, or yet consisting of, passaging iPSCs as small colonies at 15-20% confluence from day 0 to day 1. In one aspect, the iPSCs are derived from adult somatic cells such as a human skin fibroblast, or a human mesenchymal stem cell.

In another aspect, the method further comprises, or consists essentially of, or yet consists of replacing the culture medium on day 1 with GIBCO Neural Induction Medium that are cultured in the absence of feeder cells. In another aspect, the method further comprises, or consists essentially of, or yet further consists of, complete media changes of the cells on days 3 and day 5 with GIBCO Neural Induction Medium, the cells being cultured in the absence of feeder cells.

In a further aspect, the method further comprises, or consists essentially of, or yet consists of removing non-neuronal differentiated cells using methods known in the art, e.g., by removing the cells by use of cell markers that identify either the neuronal differentiated cells and/or removing cells that express non-neuronal differentiation markers. In a further aspect the removing is done manually.

In another embodiment, the method further comprises, or consists essentially of, or yet further consists of, harvesting (e.g., isolating) NSCs using Accutase on day 7 and expanding the cells on poly-O-laminin-coated plates with Neural Expansion Medium (NSC expansion medium), the method steps being performed in the absence of feeder cells. In another aspect, the method further comprises, or consists essentially of, or yet further consists of, confirming the generation of NSCs by immunohistochemistry for expression of markers of NSCs. Non-limiting examples of such markers comprise NESTIN and SOX2.

This disclosure also provides a method of producing human astrocytes from human neural stem cells, comprising, or consisting essentially of, or yet further consisting of culturing NSCs obtained as described above in culture media that comprises, or consists essentially of, or yet further consists of expansion medium on Matrigel-coated tissue culture (TC) plastic to a confluence of about 80-90%. In a further aspect, the method further comprises, or consists essentially of, or yet further consists of supplementing the culture medium with ADM1 such that the ratio of NSC expansion media to ADM1 is 1:1. In a further embodiment, the method further comprises, or consists essentially of, or yet further consists of performing a 50% media change of the cell culture media with ADM1 every 2 days. In another aspect, the method further comprises, or consists essentially of, or yet further consists of passaging cells at 1:3 onto Matrigel-coated TC plastic on day 7.

In another aspect, the step of passaging comprises, or consists essentially of, or yet further consists of: rinsing the cells with pre-warmed 1× HBSS (without Mg2+ and Ca2+) 3 times; incubating with pre-warmed Accutase at 37° C. for 5 minutes; tapping plates containing the cells dislodge the cells; adding ADM1 to the Accutase at a 2:1 ratio; and collecting the cells by centrifugation at 300×g for 5 minutes at room temperature.

In a further aspect, the method further comprises, or consists essentially of, or yet further consists of comprising performing a 50% media change every 2 days. In another aspect, the method further comprises, or consists essentially of, or yet further consists of comprising passaging one-third of the cells on day 15 onto new Matrigel-coated TC plastic in ADM1:ADM2a/b at a 1:1 ratio. The method further comprises, or consists essentially of, or yet further consists of comprising passaging one-third of the cells at 30 divisions onto new Matrigel-coated TC plastic in complete Barres media. In addition, the method further comprises, or consists essentially of, or yet further consists of passaging after 3-4 days one-half of the astrocytes generated to remove neurons onto Matrigel-coated TC plastic in Barres media. In a yet further aspect, the method further comprises, or consists essentially of, or yet further consists of maturing the astrocytes.

This disclosure also provides the isolated human astrocytes or purified populations of human astrocytes prepared by the methods of this disclosure; the astrocytes being identified by the markers GFAP, AQP4 and/or S100B. In one aspect, the purified population of human astrocytes in population are at least 50%, or alternatively at least 60%, or alternatively at least 70%, or alternatively at least 75%, or alternatively at least 80%, or alternatively at least 85%, or alternatively at least 90%, or alternatively at least 95% of the cells in the population. In one embodiment, the astrocytes in the composition are detectably labeled.

Further provided are compositions comprising the astrocytes and/or population of same and a carrier, such as a pharmaceutically acceptable carrier and/or one or more of a preservative or stabilizer, that in one aspect, is non-naturally occurring.

In one aspect, the astrocytes are co-cultured with iPSC-derived microglia. In this aspect, the differentiation of both the astrocytes and the microglia are serum-free, a feature which avoids serum activation of glia and which is an important factor to prevent confounding a study. In such aspect, an astrocyte can be identified by protein markers, such as GFAP, AQP4 and S100B. Alternatively, astrocytes may be identified by their functions, such as the ability to uptake glutamate. Further, astrocytes may be identified by a range of diagnostic tests, such as their ability to increase TEER in brain microvascular endothelial cells (“BMECs”). The astrocytes can be identified among the microglia by labeling, such as by labeling the astrocytes with GFAP and the labeling the microglia with Ibal.

The astrocytes, population or compositions containing same can be used to deliver the astrocytes to a tissue, comprising contacting the tissue with the astrocytes, population, or compositions.

The astrocytes prepared by the methods can for the treatment of neurological diseases or disorders, comprising, or consisting essentially of, or yet consisting of, administering to a subject in need thereof, the isolated astrocyte or population of such thereby treating the neurological disorder. In one aspect, administration comprises direct infusion of the isolated cells or population of cells into the CNS of the subject or by intracranial injection of the cells or population of cells. In one aspect, the subject is a human patient, and the cells can be derived from cells that are allogenic or autologous to the subject receiving them.

### BRIEF DESCRIPTION OF THE DRAWINGS

FIGS. 1A-1F: Generation of iPS-derived astrocytes from patient-derived iPSCs. Generation and confirmation of the pluripotency of iPSCs (FIG. 1A) and karyotype (FIG. 1B) of iPSCs. (FIG. 1C) iPSCs are differentiated into NSCs as confirmed by immunohistochmical analysis. NSCs are characterized by the expression of Nestin and Sox-2. (FIG. 1D) Fully defined scheme for the differentiation of NSCs to astrocytes. Flow cytometry analysis indicates the expression of GFAP and AQP4 by 30 divisions (“DIV”). (FIG. 1E) Gene expression analysis (RT-qPCR) indicates the generation of mature astrocytes by 75 DIV. (FIG. 1F) Whole transcriptome PCA reveals that iAstrocytes cluster with primary fetal astrocytes.

FIGS. 2A-2I: iAstrocytes phagocytose synaptosomes, express canonical markers typical of astrocytes, including functional glutamate receptors, and can increase blood-brain barrier properties of BMECs. (FIG. 2A) Amnis Imagestream was utilized to examine phagocytosis of human synaptosomes by astrocytes. Synaptosomes were conjugated with pHrodoRed (Lifetech) as previously described (Abud et al., iPSC-Derived Human Microglia-like Cells to Study Neurological Diseases, Neuron 94(2):278-293 (2017)) and incubated with iPS-derived astrocytes. Astrocytes were then labeled with antibodies against CD44 and MERTK and CD44-positive cells were examined for the presence of pHrodoRed. (FIG. 2A) Flow cytometry analysis of astrocytes gate for synaptosome phagocytic events. (FIG. 2B) Representative images of iPS-derived astrocytes can evaluate viability and can be used to examine co-localization between synaptosome-containing cellular compartments and MERTK. (FIG. 2C) iPS astrocytes can functionally phagocytose synaptosomes to the same extent as fetal astroctyes. (FIG. 2D) iPS-derived astrocytes that demonstrate phagocytic ability express MERTK, which is a cell surface receptor essential for astrocyte-mediated synaptic pruning. (FIG. 2E) By ICC, iAstrocyte express the canonical protein markers GFAP, AQP4, and S100B is similar to or greater fetal astrocytes. (FIG. 2F) iAstrocytes exhibit the ability to uptake glutamate. (FIG. 2G) Using methods known in the art, brain microvascular endothelial cells (BMECs) were generated that express tight junction proteins CLDN5, ZO-1, and Occludin. BMECS express higher levels of APOE and the glucose transporter protein when compared to a commercial source (FIG. 2I). (FIG. 2H) iAstrocytes exhibit the ability to increase TEER in BMECs. TEER in BMECS only is increased when co-cultured with iAstrocyte. (FIG. 2I, left and right panels) graphically show transformation of BMEC-APOE (left panel) and Transformation of BMEC-GLUTI (right panel) as measured by relative expression normalized by GAPDH.

FIGS. 3A-3B: (FIG. 3A) iAstrocytes are compatible for study with microglia co-cultures. Cultured iAstrocytes are subsequently incubated with iPSC-derived microglia (Abud et al., 2017). (FIG. 3B) iAstrocytes are labeled with GFAP and iPSC-derived microglia are labeled with Iba1 (light grey). The differentiation protocols for both microglia and astrocytes are serum-free and thus, make them compatible to study together without the confounds of serum activation of glia. Astrocytes phagocytose pHrodoRed-labeled myelin. Over the course of 24 h, myelin was incubated with iAstrocytes and images captured every hour and demonstrates that iAstrocytes can be used to study efferocytosis

FIGS. 4A-4D: TGFβ is necessary for quiescent astrocytes. iAstrocytes demonstrate phagocytosis of myelin which is inhibited by TGFβ. Time-lapse imaging reveals myelin phagocytosis. (FIGS. 4A-4C, FIG. 4A: 0 hour; FIG. 4B, 12 hours; and FIG. 4C, 24 hours). Phagocytosis can be visualized by pHrodoRed internalization which is absent at time zero (FIG. 4A). Phagocytosis of pHrodoRed-conjugated myelin is detected in iAstrocytes at 12 h (FIG. 4B), which gradually increases with time (24 h, FIG. 4C). FIG. 4D graphically depicts total red object area (y-axis) versus time in hours (x-axis).

FIGS. 5A-5L: iAstrocytes functionally incorporate within transgenic mice brains. (FIGS. 5A-5L) iAstrocytes transplanted into xenotransplantation compatible mice survive. Transplanted iAstrocytes are visualized with the human specific markers Ku80 (FIG. 5A, FIG. 5E, FIG. 5I) and STEM123 (FIG. 5B, FIG. 5F, FIG. 5J) at different magnifications. These markers distinguish transplanted cells from endogenous astrocytes which are labeled by a non-species specific GFAP (FIG. 5C, FIG. 5G, FIG. 5K). Ku80 co-localized with cells that express STEM123 (FIG. 5D, FIG. 5H, FIG. 5L).

FIGS. 6A-6E: iPSC-derived astrocytes functionally promote mature network synchronous bursting of neurons. Compared to primary astrocytes, iPSC-derived astrocytes (iAstrocytes) promote the electrophysiological maturation of neurons as assessed by multi-electrodes arrays (MEA). Neurons cultured with iPSC-derived astrocytes (iAstrocytes) up to 35 DIV, display firing as assessed by the MEA parameters, wMFR (weighted mean firing rate), burst frequency (Hz), synchrony index, network burst frequency and interspike interval (ISI) variance. (FIG. 6A) wMFR (Hz) versus DPP. (FIG. 6B) Burst Frequency (Hz) versus DPP. (FIG. 6C) measured Synchrony index versus DPP. (FIG. 6D) measured ISI CoV versus DPP. (FIG. 6E) shows network burst frequency (Hz) versus DPP.

### DETAILED DESCRIPTION

Astrocytes are the most abundant central nervous system cell type and have been implicated in the pathobiology of many neurological diseases. To properly study human astrocyte biology and their role in disease requires astrocyte isolation from human primary tissue, which is both a limiting resource and highly variable due to the source of material. Therefore, the identification and validation of drug targets to modulate astrocyte function are difficult to achieve with current methodologies to isolate primary cells to study astrocyte biology.

While examples of methods of generation of human astrocytes are known, these methods have several imitations and disadvantages as described herein.

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

Throughout and within this application technical and patent literature are referenced by a citation. For certain of these references, the identifying citation is found at the end of this application immediately preceding the claims. All publications are incorporated by reference into the present disclosure to more fully describe the state of the art to which this disclosure pertains.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As used herein, the term “completeTeSR-E8 medium” refers to a feeder-free, animal component-free culture medium for maintenance of human ES and iPS cells. The medium is commercially available from a number of vendors, for example, Stem Cell Technologies (https://www.stemcell.com/products/tesr-e8.html?gclid=EAIaIQobChMI9J6j1Lji_wIVwhd9Ch2ZygC2EAAYAyAAEgJMtvD_BwE, last accessed on Jun. 26, 2023) and ThermoFisher Scienific (https://www.thermofisher.com/order/catalog/product/A1517001 last accessed on Jun. 26, 2023).

As used herein, the term “confluent population” intends a population of cells that are in contiguous contact with the adjacent cells.

An “ultra-low attachment surface” intends cell or tissue culture surfaces that in some aspects contain a covalently bound hydrogel layer that is hydrophilic and neutrally charged. Since proteins and other biomolecules passively adsorb to polystyrene surfaces through either hydrophobic or ionic interactions, this hydrogel surface naturally inhibits nonspecific immobilization via these forces, thus inhibiting subsequent cell attachment. These surfaces are commercially available from a variety of vendors, e.g. Millipore-Sigma, Fisher-Scientific, and S-bio. Methods are known in the art for manufacturing cell culture plates and surfaces.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

An equivalent or biological equivalent nucleic acid, polynucleotide or oligonucleotide or peptide is one having at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, oligonucleotide or peptide.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In some embodiments, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-histidine (His) tag, calmodulin binding protein (CBP), or maltose-binding protein (MBP)), or a fluorescent tag.

The term “propagate” or “expand” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

A population of cells intends a collection of more than one cell that is identical (clonal) or non-identical in phenotype and/or genotype. A substantially homogenous population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers.

The term “stem cell” refers to a cell that is in an undifferentiated or partially differentiated state and has the capacity for self-renewal or to generate differentiated progeny or both. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural or neuronal stem cells (NSCs). In some embodiments, the stem or progenitor cells can be embryonic stem cells or an induced pluripotent stem cell (iPSC). In some embodiments, the stem or progenitor cells are hematopoietic stem cells (HSCs).

As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

As used herein, a “pluripotent cell” defines a less differentiated cell that can give rise to at least two distinct (genotypically or phenotypically or both) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

An “induced pluripotent cell” intends embryonic-like cells reprogrammed to the immature phenotype from adult cells. Various methods are known in the art, e.g., “A simple new way to induce pluripotency: Acid.” Nature, 29 Jan. 2014 and available at sciencedaily.com/releases/2014/01/140129184445, last accessed on Feb. 5, 2014 and U.S. Patent Application Publication No. 2010/0041054. Human iPSCs also express stem cell markers and can generate cells characteristic of all three germ layers.

A neural stem cell is a cell that can be isolated from the adult central nervous systems of mammals, including humans. They have been shown to generate neurons, migrate and send out axonal and dendritic projections and integrate into pre-existing neuronal circuits and contribute to normal brain function. Reviews of research in this area are found in Miller (2006) The Promise of Stem Cells for Neural Repair, Brain Res. Vol. 1091(1):258-264; Pluchino et al. (2005) Neural Stem Cells and Their Use as Therapeutic Tool in Neurological Disorders, Brain Res. Brain Res. Rev., Vol. 48(2):211-219; and Goh, et al. (2003) Adult Neural Stem Cells and Repair of the Adult Central Nervous System, J. Hematother. Stem Cell Res., Vol. 12(6):671-679.

An “astrocyte” is a subtype of glial cells that make up the majority of cells in the human central nervous system (CNS). They perform metabolic, structural, homeostatic, and neuroprotective tasks such as clearing excess neurotransmitters, stabilizing and regulating the blood-brain barrier, and promoting synapse formation. An “iAstrocyte” is an astrocyte prepared by the method of this disclosure. iAstrocytes are identified by one or more markers set forth in the Brief Description of the Figures and experimental methods. For example, iAstrocyte express the canonical protein markers GFAP, AQP4, and S100. iAstrocytes exhibit the ability to uptake glutamate. iAstrocytes exhibit the ability to increase TEER in BMECs.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal, or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multi-lineage stem cell is a myogenic cell and an adipogenic cell (both are of mesodermal origin yet give rise to different tissues). Another example is a neurogenic cell (of ectodermal origin) and adipogenic cell (of mesodermal origin).

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

“Embryoid bodies or EBs” are three-dimensional (3D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

A “composition” is intended to mean a combination of active polypeptide, polynucleotide or antibody and another compound or composition, inert (e.g. a detectable label) or active (e.g. a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like which is susceptible to neurodegenerative disease. In one embodiment, the mammals include horses, dogs, and cats. In another embodiment of the present invention, the human is an adolescent or infant under the age of eighteen years of age.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication ex vivo, in vitro or in vivo.

As used herein, the term “contacting” means direct or indirect binding or interaction between two or more molecules or other entities. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository), intracranial, or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

A “neurodegenerative disease or disorder” is a disease or phenotype characterized by degeneration of the nervous system, especially the neurons in the CNS. Non-limiting examples of such include a central nervous system (CNS) disease or disorder. A CNS disease or disorder is a group of neurological disorders that affect the structure of function of the brain or spinal cord, and that may result in degeneration of one or more parts of the brain or spinal cord. Non-limiting examples include Huntington's disease (HD), Alzheimer's disease, Parkinson's disease, traumatic brain injury, stroke, autoimmune disorders such as multiple sclerosis, primary or secondary progressive multiple sclerosis, relapsing remitting multiple sclerosis, brain inflammation, Bell's palsy, cervical spondylosis, carpal tunnel syndrome, brain or spinal cord tumors, peripheral neuropathy, Guillain-Barre syndrome, amyotrophic lateral sclerosis, and Huntington chorea. To treat or ameliorate the symptoms of a CNS injury intends to improve the patient's nerve function reduce the adverse effect of inherited or acquired disease, injury or a disorder. The symptoms and course of the disease are known to the skilled artisan, see, hopkinsmedicine.org/healthlibrary/conditions/nervous_system_disorders/overview_of_nervous_system_disorders_85,P00799, accessed on May 21, 2018.

“To enhance synaptic connections” intends to promote connections between neurons or neuronal receptors.

A synapse is a junction between two nerve cells, consisting of a minute gap across which impulses pass by diffusion of a neurotransmitter.

The term “feeder cell” indicates a cell which is part of a cell culture but that is not capable of dividing. The feeder cells secrete molecules which aid in the growth of a different cell type also present in the cell culture. Feeder cells are distinct from a co-culture system due to the fact that the feeder cells are incapable of proliferation.

The term “non-neuronal differentiated cells” indicates cells which are not neural cells and that have undergone differentiation. Differentiation occurs when immature, unspecialized cells take on new characteristics as they mature. These new characteristics often give the cells their unique functions.

The term “Poly-L-laminin-coated plates” indicates cell culture plates which have been coated with Poly-L-Ornithine, a synthetic amino acid, and laminin. Poly-L-Ornithine is a positively charged amino acid, which can be beneficial in increasing cell attachment to cell culture plates. Combining Poly-L-Ornithine with laminins, a glycoprotein, is often used for neuronal growth and cellular differentiation. Poly-L-laminin coated plates can be purchased through a number of companies, such as Corning. See https://ecatalog.corning.com/life-sciences/b2c/US/en/Surfaces/Biologically-Coated-Surfaces/Poly-D-Lysine-Laminin/Corning%C2%AE-BioCoat%C2%AE-Poly-L-Ornithine-Laminin-Plates/p/corningBioCoatPolyLOrnithineLamininPlates, last visited Jun. 28, 2023.

The term “neural expansion Medium” indicates cellular growth medium that is designed to aid in the proliferation of neural stem cells. Neural expansion medium is one of the many components used to grow and differentiate cells. It is available from a number of suppliers, such as Millipore Sigma or Sigma-Aldrich. See https://www.sigmaaldrich.com/US/en/product/sigma/s3194, last visited Jun. 28, 2023.

The term “ADM” as in “ADM1” and “ADM2a/b” refers to astrocyte differentiation medium. This is a medium designed to differentiate cell cultures into astrocytes. ADM often contains a variety of components, such as IGF-1, Fetal bovine serum, and other supplements to impact cell growth. ADM is commercially available from ScienCell Research Laboratories, and Thermo Fisher Scientific.

The term “1× HBSS” refers to Hanks' Balanced Salt Solution. This solution is used for a variety of cell culture methods. The medium is used to provide water to cells, as well as certain essential, non-organic ions. The term “1×” indicated the concentration of the solution used. 1× HBSS is available for purchase from several suppliers, such as Thermo Fisher and ATCC. See https://www.thermofisher.com/order/catalog/product/14025092, last visited Jun. 28, 2023.

The term “maturing the astrocytes” indicates the methods by which astrocytes can become differentiated. Mature astrocytes have undergone the process of differentiation, which changes their capacity to perform certain functions. Astrocytes are prompted to mature in several ways, including via extracellular signals.

The term “iAstrocytes” means astrocytes which are the result of artificial cellular differentiation. An immature cell, when exposed to certain chemicals, cellular signals and/or other stimuli, can differentiate into an astrocyte. The immature cell used is often an iPSC. When this is done in vitro, the resulting astrocytes are referenced using the term iAstrocyte.

The term “CD 44” indicates a particular type of cell surface receptor. The receptor is often highly expressed in certain forms of cancers. The receptor is responsible, in part, for regulating metastasis through its interaction with certain extracellular ligands. Additional information on the protein structure and amino acid sequence can be found at https://www.frontiersin.org/articles/10.3389/fcell.2017.00018/full, last visited Jun. 28, 2023.

The term “MERTK” indicates a particular receptor kinase. The receptor responds to a number of ligands found in the extracellular matrix and is responsible in part for differentiation and phagocytosis. Additional information and amino acid sequences can be found at https://www.uniprot.org/uniprotkb/Q12866/entry, last visited Jun. 28, 2023.

The term “canonical protein markers”, and references to “GFAP, AQP4 and S100B”, indicates proteins which are commonly used to identify mature astrocytes. “GFAP” or Glial Fibrillary Acidic Protein is one of the most used markers to identify astroglia activation. “AQP4” or Aquaporin-4 is also used to identify astrocyte differentiation, as is the calcium binding protein “S100B”. Additional information on protein structure and amino acid sequence can be found at: https://www.genecards.org/cgi-bin/carddisp.pl?gene=S100B; https://www.uniprot.org/uniprotkb/P55087/entry; and https://www.uniprot.org/uniprotkb/P14136/entry, last visited Jun. 28, 2023.

The term “tight junction proteins”, and examples such as “CLDN5”, “ZO-1” and “Occludin”, indicates proteins which can seal the space between cells. “CLDN5” is a claudin protein and plays a role as a membrane protein and a component of a tight junction. “ZO-1” is a scaffold protein, with a role in both cell adhesion and the regulation of molecule movement between cells. Occludin is a membrane linker protein, and similarly plays a role in maintaining the tight junctions between cells. Additional information on protein structure and amino acid sequence can be found at: https://www.uniprot.org/uniprotkb/D3DX19/entry; https://www.ncbi.nlm.nih.gov/gene/7082; and https://www.uniprot.org/uniprotkb/A0A0G2JMZ8/entry, last visited Jun. 28, 2023.

The term “APOE” refers to a specific apolipoprotein. These proteins often associate with lipids and play a large role in mediating lipid transport. Additional information on protein structure and amino acid sequence can be found at: https://www.uniprot.org/uniprotkb/P02649/entry, last visited June 38, 2023.

The term “TEER” refers to transepithelial/transendothelial electrical resistance. TEER is a widely used method of determining and measuring tight junction integrity within cell cultures and models. The technique is often used to determine how easily a molecule or pharmaceutical will be able to penetrate a barrier of cells.

The term “Ku80” refers to a specific antibody which bind the human Ku protein. The protein is highly conserved, indicating that it will be present in most, if not all, human cells. The Ku80 antibody marker is available for purchase through companies such as BioRad and Biotechne. See https://www.bio-rad-antibodies.com/polyclonal/human-ku80-antibody-ahp1391.html?f=purified&JSESSIONID_STERLING=E705EA9E7C4487646066389FB7DE8D3E.ecommerce1&evCntryLang=US-en&cntry=US&thirdPartyCookieEnabled=true, last visited Jun. 28, 2023.

The term “STEM123” refers to a human specific antibody that is often used to detect differentiated astrocytes. The marker is available for purchase from companies such as Fisher Scientific and Takara. See https://www.fishersci.com/shop/products/stem 123-50ug/NC2323730, last visited Jun. 28, 2023.

The term “GFAP”, or glial fibrillary acidic protein, refers to a specific intermediate filament protein. The function of these proteins often relates to the cytoskeletal structure of glia cells. Specific antibodies can bind to the GFAP protein for labeling purposes. Additional information on amino acid sequence or protein structure can be found at https://www.uniprot.org/uniprotkb/P14136/entry, last visited Jun. 28, 2023.

Further provided are compositions comprising the astrocytes and/or population of same and a carrier, such as a pharmaceutically acceptable carrier and/or one or more of a preservative or stabilizer, that in one aspect, is non-naturally occurring.

In one aspect, the astrocytes are co-cultured with iPSC-derived microglia. In this aspect, the differentiation of both the astrocytes and the microglia are serum-free, a feature which avoids serum activation of glia and which is an important factor to prevent confounding a study. In such aspect, an astrocyte can be identified by protein markers, such as GFAP, AQP4 and S100B. Alternatively, astrocytes may be identified by their functions, such as the ability to uptake glutamate. Further, astrocytes may be identified by a range of diagnostic tests, such as their ability to increase TEER in brain microvascular endothelial cells (“BMECs”). The astrocytes can be identified among the microglia by labeling, such as by labeling the astrocytes with GFAP and the labeling the microglia with Ibal.

The astrocytes, population or compositions containing same can be used to deliver the astrocytes to a tissue, comprising contacting the tissue with the astrocytes, population, or compositions.

The astrocytes prepared by the methods can for the treatment of neurological diseases or disorders, comprising, or consisting essentially of, or yet consisting of, administering to a subject in need thereof, the isolated astrocyte or population of such thereby treating the neurological disorder. In one aspect, administration comprises direct infusion of the isolated cells or population of cells into the CNS of the subject or by intracranial injection of the cells or population of cells. In one aspect, the subject is a human patient, and the cells can be derived from cells that are allogenic or autologous to the subject receiving them.

### Modes for Carrying Out the Disclosure

In some embodiments, a method of producing human astrocytes from neural stem cells (NSCs) is provided. In another aspect, this disclosure provides a method for generating human astrocytes from human neural stem cells produced from human induced pluripotent stem cells (iPSCs). Applicant has found that the cells produced by these methods produce human astrocytes that function as mature neurons, as assessed by electrophysiology. The human astrocytes produced by the method of this disclosure have promote blood brain barrier (BBB) function, can engraft in brain, uptake glutamate, and phagocytose.

In some embodiments, the method of producing human astrocytes from NSCs comprises providing induced progenitor cells (iPSCs), differentiating iPSCs to NSCs, and differentiating NSCs to astrocytes.

In some embodiments of the method of producing human astrocytes from NSCs, differentiating iPSCs to NSCs comprises culturing iPSCs in feeder-free conditions using completeTeSR-E8 medium.

In some embodiments, the method of producing human astrocytes from NSCs further comprises passaging iPSCs as small colonies at 15-20% confluence (day 0). As used herein, the term “passaging” intends established a new cell culture made by transferring some or all cells from a previous culture to fresh growth medium.

In some embodiments, the method of producing human astrocytes from NSCs further comprises replacing the culture medium on day 1 with GIBCO Neural Induction Medium, commercially available from various vendors such as Fisher Scientific, see https://www.fishersci.com/shop/products/psc-neural-induction-medium/A1647801, last accessed on Jun. 26, 2023.

In some embodiments, the method of producing human astrocytes from NSCs further comprises complete media changes on days 3 and day 5.

In some embodiments, the method of producing human astrocytes from NSCs further comprises manually removing non-neuronal differentiated cells using methods known in the art, such as the use of cell specific markers.

In some embodiments, the method of producing human astrocytes from NSCs further comprises harvesting NSCs using Accutase on day 7 and expanding on poly-O-laminin-coated plates with Neural Expansion Medium (Gibco). Accutase is commercially available from Innovative Cell Technologies, Inc. (http://www.accutase.com/accutase.html), last accessed on Jun. 26, 2023. Neural Expansion Medium is commercially available from (Gibco), (https://www.sigmaaldrich.com/US/en/product/mm/scm004, last accessed on Jun. 26, 2023).

In some embodiments, the method of producing human astrocytes from NSCs comprises confirming the generation of NSCs by immunohistochemistry for expression of markers of NSCs.

In some embodiments of the method of producing human astrocytes from NSCs, the markers of NSCs comprise NESTIN and SOX2.

In some embodiments of the method of producing human astrocytes from NSCs, differentiating NSCs to astrocytes comprises culturing NSCs obtained in the step of differentiating iPSCs to NSCs in NSC expansion medium on Matrigel-coated tissue culture “TC” plastic to a confluence of about 80-90%. Methods to prepare Matrigel coated tissue culture plates are known in the art, or can be commercially available from various vendors such as Thermo Fisher Scientific.

In some embodiments, the method of producing human astrocytes from NSCs comprises supplementing with ADM1 such that the ratio of NSC expansion media to ADM1 is 1:1.

In some embodiments, the method of producing human astrocytes from NSCs comprises performing a 50% media change with ADM1 every 2 days.

In some embodiments, the method of producing human astrocytes from NSCs comprises passaging cells at 1:3 onto Matrigel-coated TC plastic on day 7.

In some embodiments of the method of producing human astrocytes from NSCs, the passaging comprises rinsing the cells with pre-warmed 1× HBSS (without Mg2+ and Ca2+) 3 times, incubating with pre-warmed Accutase at 37° C. for 5 minutes, tapping plates containing the cells dislodge the cells, adding ADM1 to the Accutase at a 2:1 ratio, and collecting the cells by centrifugation at 300×g for 5 minutes at room temperature.

In some embodiments, the method of producing human astrocytes from NSCs comprises performing a 50% media change every 2 days.

In some embodiments, the method of producing human astrocytes from NSCs comprises passaging one-third of the cells on day 15 onto new Matrigel-coated TC plastic in ADM1:ADM2a/b at a 1:1 ratio.

In some embodiments, the method of producing human astrocytes from NSCs comprises passaging one-third of the cells at 30 divisions onto new Matrigel-coated TC plastic in complete Barres media.

In some embodiments, the method of producing human astrocytes from NSCs comprises passaging after 3-4 days one-half of the astrocytes generated to remove neurons onto Matrigel-coated TC plastic in Barres media.

In some embodiments, the method of producing human astrocytes from NSCs comprises further maturing the astrocytes.

This disclosure also provides the isolated human astrocytes or purified populations of human astrocytes prepared by the methods of this disclosure; the astrocytes being identified by the markers GFAP, AQP4 and/or S100B. In one aspect, the purified population of human astrocytes in population are at least 50%, or alternatively at least 60%, or alternatively at least 70%, or alternatively at least 75%, or alternatively at least 80%, or alternatively at least 85%, or alternatively at least 90%, or alternatively at least 95% of the cells in the population. In one embodiment, the astrocytes in the composition are detectably labeled or labeled with a purification marker.

Further provided are compositions comprising the astrocytes and/or population of same and a carrier, such as a pharmaceutically acceptable carrier and/or one or more of a preservative or stabilizer, that in one aspect, is non-naturally occurring. Non-limiting examples include any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton). The term pharmaceutically acceptable carrier (or medium), which may be used interchangeably with the term biologically compatible carrier or medium, refers to reagents, cells, compounds, materials, compositions, or dosage forms, or any combination thereof, that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit to risk ratio.

Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodible). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result or protection or both desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

In one aspect, the astrocytes are co-cultured with iPSC-derived microglia. In this aspect, the differentiation of both the astrocytes and the microglia are serum-free, a feature which avoids serum activation of glia and which is an important factor to prevent confounding a study. In such aspect, an astrocyte can be identified by protein markers, such as GFAP, AQP4 and S100B. Alternatively, astrocytes may be identified by their functions, such as the ability to uptake glutamate. Further, astrocytes may be identified by a range of diagnostic tests, such as their ability to increase TEER in brain microvascular endothelial cells (“BMECs”). The astrocytes can be identified among the microglia by labeling, such as by labeling the astrocytes with GFAP and the labeling the microglia with Iba1.

The astrocytes, population or compositions containing same can be used to deliver the astrocytes to a tissue, comprising contacting the tissue with the astrocytes, population, or compositions.

The astrocytes prepared by the methods can for the treatment of neurological diseases or disorders, comprising, or consisting essentially of, or yet consisting of, administering to a subject in need thereof, the isolated astrocyte or population of such thereby treating the neurological disorder. In one aspect, administration comprises direct infusion of the isolated cells or population of cells into the CNS of the subject or by intracranial injection of the cells or population of cells. In one aspect, the subject is a human patient, and the cells can be derived from cells that are allogenic or autologous to the subject receiving them. Administration can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an intratumoral administration, or administration to a tumor microenvironment, or both. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

Administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

In some embodiments, 1×10⁴ to 1×10¹⁵ or ranges in between of cells as disclosed herein are administrated to a subject, such as 1×10⁷ to 1×10¹⁰. In some embodiments, administering or a grammatical variation thereof also refers to more than one doses with certain interval. In some embodiments, the interval is 1 day, 2 days, 3 days, 4 days, 5days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. For example, cells as disclosed herein may be administered to a subject weekly and for up to four weeks.

### Experimental

### Culturing Materials and Methods

iAstrocyte Maturation and Differention: The materials required to differentiate iPSC Astrocystes include: DMEM high glucose (stored at 4° C.), BrainPhys media (stored at 4° C.), Neurobasal media (stored at 4° C.), Advanced DMEM/F12 (stored at 4° C.), Heat inactivated fetal-bovine serum (stored at −20° C.), N2 Supplement (stored at −20° C.), Glutamax (stored at 4° C.), Sodium Pyruvate (stored at 4° C.), Neurocult SM1 supplement (stored at −20° C.), N-acetylcysteine (stored at −20° C.), rhuEBGF (stored at −20° C.), 1× HBSS (stored at 4° C.), Accutase (stored at −20° C.), Matrigel (stored at −80° C.), TC-Treated T75s (stored at room temperature), and SATO aliquots (stored at −20° C.). These materials are commercially available, for example from Sigma or Thermo Fisher.

Astrocyte Differentiation medium (“ADM”) #1 includes: DMEM, N2 supplement at a concentration of 1×, Glutamax at a concentration of 1×, LIF at a concentration of 100 ng/mL, CNTF at a concentration of 100 ng/ml, and bFGF at a concentration of 50 ng/mL. Each of the previously listed components is added after filtering through a 0.22 μm filter, except for the DMEM and the Glutamax. The components LIF, CNTF, and bFGF are added after the filtering has been completed, but before cell feeding.

ADM #2a includes: DMEM at a concentration of 50%, Neurobasal at a concentration of 50%, Glutamax at a concentration of 1×, and Sodium Pyruvate at a concentration of 1×. Additionally, SATO is added at a concentration of 1×, following filtering through a 0.22 μm filter. The final three components, LIF at a concentration of 25 ng/ml, CNTF at a concentration of 50 ng/ml and bFGF at a concentration of 50 ng/ml, are added after filtering through a 0.22 μm filter, but before feeding.

ADM #2b includes: BrainPhys, as well as Neurocult SM1 at a concentration of 1×, LIF at a concentration of 25 ng/mL, CNTF at a concentration of 50 ng/ml and bFGF at a concentration of 50 ng/mL. All components, except BrainPhys, are added after filtering through a 0.22 μm filter, but just before feeding.

ADM #3, otherwise known as Barres medium for maturation includes: DMEM at a concentration of 50%, Neurobasal at a concentration of 50%, Glutamax at a concentration of 1×, and Sodium Pyruvate at a concentration of 1×. Additionally, SATO at a concentration of 1×, NAC at a concentration of 5 ng/ml and hEBGF at a concentration of 5 ng/ml are all added after filtering through a 0.22 μm filter. The NAC and hEBGF are added just before feeding.

For each media, cytokines are to added fresh before use. Additionally, it is recommended to base media in 50 mL and to prep the cytokines in 1000× concentration. This ensures quick use in 50-100 mL of medium.

For the present example of differentiating iPSC astrocytes, the 10318 cell line performed was used (see e.g., American Type Culture Collection “ATCC”), with T75s. The method starts with the NSCs P7, which are derived from the 10318 cell line. When the following methods are used with T75s, these cells differentiate into astrocytes.

To begin differentiation, confluent NSCs, which have been cultured on Matrigel-coated T75s for a period of 7-8 days, are added to a flask with ADM1, in a ratio of 50/50. This mixture becomes NSC media/ADM1 and will have a final volume of roughly 20 mL. A 50% media-change can be performed every 2 days following creation, using ADM1 media.

After 7 days, the passaging process can begin. The cells can be passaged into Matrigel-coated T75s at a ratio of 1:3. The cells are then passaged by washing with warm 1× HBSS three times. This HBSS can be free of Mg²⁻ and Ca²⁺. The wash can be aspirated to waste. Then 5 mL of pre-warmed accutase can be added to each T75, and the plate can be placed under an incubator for five minutes. Following this, the T75 can be lightly tapped in the hood to gently dislodge the cells. Once this is complete, 10 mL of ADM1 can be quickly added to each flask to collect the cells. The amount of ADM1 may change but can be in a ratio of 2:1 compared to the Accutase. The collected cells can then be centrifuged at 300×g for five minutes at room temperature.

Following the use of the centrifuge, 50% media changes can continue to be performed every two days. This is done to ensure that each flask maintains a total volume of roughly 20 mL. On day 15, the cells once again undergo the passaging process described above. However, the media used for the day 15 passage is a 50/50 mixture of ADM1 and ADM2a/b. The same passaging process is preformed again on day 30, this time using Barres media. Roughly three to four days later, the cells are passaged for a final time in order to remove the neurons to Matrigel-coated T75s. Alternatively, they can be removed to 6-wells using a ratio of 1:2 Barres Media.

At this point, the iPSC astrocyte differentiation process is complete and the isolation of the cells can be determined. Additionally, it is at this point that their morphology can be assessed. This process allows for differentiation to be completed in a time span of roughly 30 to 40 days total.

### Methods of Producing Astrocytes

In some embodiments, a method of producing human astrocytes from neural stem cells (NSCs) is provided.

In some embodiments, the method comprises providing induced progenitor cells (iPSCs), differentiating iPSCs to neural stem cells (NSCs), and differentiating NSCs to astrocytes.

In some embodiments, differentiating iPSCs to NSCs comprises culturing iPSCs in feeder-free conditions using completeTeSR-E8 medium.

In some embodiments, differentiating iPSCs to NSCs further comprises passaging iPSCs as small colonies at 15-20% confluence (day 0). In some embodiments, the confluence is about 7.5, 10, 12.5, 15, 17.5, 20, 22.5, 25, 27.5, 30, 32.5, 35, 37.5, or 40%.

In some embodiments, differentiating iPSCs to NSCs further comprises replacing the culture medium on day 1 with GIBCO Neural Induction Medium. In some embodiments, the culture medium is replaced on day 1.5, day 2, day 2.5, or day 3.

In some embodiments, differentiating iPSCs to NSCs further comprises complete media changes on day 3 and day 5. In some embodiments, complete media changes are performed on day 1, 2, 3, 4, 5, 6, and/or 7.

In some embodiments, differentiating iPSCs to NSCs further comprises manually removing non-neuronal differentiated cells.

In some embodiments, differentiating iPSCs to NSCs further comprises harvesting NSCs using Accutase on day 7 and expanding on poly-O-laminin-coated plates with Neural Expansion Medium (Gibco). In some embodiments, the harvesting is performed on day 5, day 6, day 7, day 8, or day 9.

In some embodiments, differentiating iPSCs to NSCs comprises confirming the generation of NSCs by immunohistochemistry for expression of markers of NSCs.

In some embodiments, the markers of NSCs comprise NESTIN and SOX2.

In some embodiments, differentiating NSCs to astrocytes comprises culturing NSCs obtained by differentiating iPSCs to NSCs in NSC expansion medium on Matrigel-coated TC plastic to a confluence of about 80-90%. In some embodiments, the confluence is about 50, 55, 60, 65, 70, 75, 80, 85, 90, 91, 92, 93, 94, 95,96, 97, 98, 99, or 100%

In some embodiments, differentiating NSCs to astrocytes further comprises supplementing with ADM1 such that the ratio of NSC expansion media to ADM1 is 1:1. In some embodiments, the ratio of NSC expansion media to ADM1 is 0.25:1, 0.5:1, 0.75:1, 1:1, 1.5:1, 2:1, 2.5:1, 3:1, 3.5:1 or 4:1.

In some embodiments, differentiating NSCs to astrocytes further comprises performing a 50% media change with ADM1 every 2 days. In some embodiments, differentiating NSCs to astrocytes further comprises performing a 25, 30, 35, 40, 45, 50, 55, 60, 65, 70, 75, 80, 85, 90, or 100% media change with ADM1. In some embodiments, differentiating NSCs to astrocytes further comprises performing a media change with ADM1 every 1, 1.5, 2, 2.5, 3, 3.5, or 4 days.

In some embodiments, differentiating NSCs to astrocytes further comprises passaging one third of the cells onto Matrigel-coated TC plastic on day 7. In some embodiments, differentiating NSCs to astrocytes comprises passaging one fourth, one-fifth, one-sixth, one-seventh, one-eighth, one-ninth, or one-tenth of the cells. In some embodiments, the passaging is performed on day 4, day 5, day 6, day 7, day 8, day 9, or day 10.

In some embodiments of differentiating NSCs to astrocytes, the passaging comprises rinsing the cells with pre-warmed 1× HBSS (without Mg2+ and Ca2+) 3 times, incubating with pre-warmed Accutase at 37° C. for 5 minutes, tapping plates containing the cells dislodge the cells, adding ADM1 to the Accutase at a 2:1 ratio, and collecting the cells by centrifugation at 300×g for 5 minutes at room temperature. In some embodiments, the ADM1: Accutase ratio is about 0.25:1, 0.5:1, 0.75:1, 1:1, 1.5:1, 2:1, 2.5:1, 3:1, 3.5:1 or 4:1.

In some embodiments, differentiating NSCs to astrocytes further comprises performing a 50% media change every 2 days. In some embodiments, differentiating NSCs to astrocytes further comprises performing a 25, 30, 35, 40, 45, 50, 55, 60, 65, 70, 75, 80, 85, 90, or 100% media change. In some embodiments, differentiating NSCs to astrocytes further comprises performing a media change every 1, 1.5, 2, 2.5, 3, 3.5, or 4 days.

In some embodiments, differentiating NSCs to astrocytes further comprises passaging one-third of the cells on day 15 onto new Matrigel-coated TC plastic in ADM1:ADM2a/b at a 1:1 ratio. In some embodiments, differentiating NSCs to astrocytes further comprises passaging one fourth, one-fifth, one-sixth, one-seventh, one-eighth, one-ninth, or one-tenth of the cells. In some embodiments, differentiating NSCs to astrocytes further comprises passaging the cells on day 10, 11, 12, 13, 14, 15, 16, 17, 18, 19, or 20. In some embodiments, the ratio of ADM1:ADM2a/b is 1:0.25, 1:0.5, 1:0.75, 1:1, 1:2, 1:3, or 1:4. In some embodiments, the ratio of ADM2a/b:ADM1 is 1:0.25, 1:0.5, 1:0.75, 1:1, 1:2, 1:3, or 1:4.

In some embodiments, differentiating NSCs to astrocytes further comprises passaging one-third of the cells at 30 divisions onto new Matrigel-coated TC plastic in complete Barres media. In some embodiments, differentiating NSCs to astrocytes further comprises passaging the cells at 15, 20, 25, 30, 35, 40, 45, 50, 55, or 60 divisions. In some embodiments, differentiating NSCs to astrocytes further comprises passaging one fourth, one-fifth, one-sixth, one-seventh, one-eighth, one-ninth, or one-tenth of the cells.

In some embodiments, differentiating NSCs to astrocytes further comprises passaging after 3-4 days one-half of the astrocytes generated to remove neurons onto Matrigel-coated TC plastic in Barres media. In some embodiments, differentiating NSCs to astrocytes further comprises passaging the astrocytes after 1-3 days. In some embodiments, differentiating NSCs to astrocytes further comprises passaging the astrocytes after 3-6 days. In some embodiments, differentiating NSCs to astrocytes further comprises passaging the astrocytes after 6-9 days. In some embodiments, differentiating NSCs to astrocytes further comprises passaging about one-half of the astrocytes. In some embodiments, differentiating NSCs to astrocytes further comprises passaging about one-third, one fourth, one-fifth, one-sixth, one-seventh, one-eighth, one-ninth, or one-tenth of the astrocytes.

In some embodiments, differentiating NSCs to astrocytes further comprises further maturing the astrocytes. In some embodiments, differentiating NSCs to astrocytes further comprises further maturing the astrocytes for about 1-3 days. In some embodiments, differentiating NSCs to astrocytes further comprises further maturing the astrocytes for about 3-6 days. In some embodiments, differentiating NSCs to astrocytes further comprises further maturing the astrocytes for about 6-9 days. In some embodiments, differentiating NSCs to astrocytes further comprises further maturing the astrocytes for about 1, 2, 3, 4, 5, 6, 7, 8, or 9 days.

### Advantages

The present invention offers several significant advantages. Non-limiting advantages of the embodiments of the methods disclosed herein include:

Demonstrated reproducibility of the method across multiple cell lines.

The method does not utilize serum (i.e., is serum free).

Incorporation of neurons in the initial steps of differentiation to generate functionally mature astrocytes. Examination of mature astrocyte markers (see Zhang et al., Purification and Characterization of Progenitor and Mature Human Astrocytes Reveals Transcriptional and Functional Differences with Mouse, Neuron 89(1): 37-53 (2015)) demonstrates the method generates mature astrocytes (AQP4 expression by qPCR).

Significantly more efficient and faster development of astrocytes utilizing a fully defined serum-free media formulation as compared to known methods. Mature functional astrocytes can be achieved in 45 divisions.

Validation of the method at the protein level by immunocytochemistry and at the whole transcriptome level by RNAseq analysis where the generation of iPSC-derived astrocytes is compared to primary astrocytes.

Comprehensive authentication of astrocyte phenotype both morphologically and with a comprehensive human whole transcriptome analysis.

Some embodiments of the methods disclosed herein relate to functional validation of astrocytes using a number of newly developed assays indicating that the methods of the present disclosure generate astrocytes that more equivalent to human brain-derived astrocytes than previous approaches. In some embodiments, the validation of astrocytes is performed using RNAseq analysis. In some embodiments, the validation of astrocytes is performed using protein expression/proteomics analysis. In some embodiments, the validation of astrocytes is performed using metabolomics analysis. In some embodiments, the validation of astrocytes is performed using genomics analysis. In some embodiments, the validation of astrocytes is performed using transcriptomics analysis. In some embodiments, the validation of astrocytes is performed using epigenomcis analysis. In some embodiments, the validation of astrocytes is performed using a combination of two or more of the foregoing omics analyses.

In some embodiments, the present disclosure demonstrates the feasibility of using human astrocyte-human neuron co-cultures to study glutamate buffering/excitotoxicity.

In some embodiments, the present disclosure demonstrates the feasibility of using human astrocyte-brain microvascular endothelial cells to study their role in blood-brain barrier function.

In some embodiments, the present disclosure demonstrates the feasibility of using human astrocyte-human microglia-like cell co-cultures to study human glia crosstalk that is only possible due to serum-free conditions, which maintain glia in a quiescent state.

In some embodiments, the astrocytes generated using embodiments of the methods disclosed herein and transplanted within xenotransplantation competent mice survive and functionally incorporate within xenotransplantation competent mice.

In some embodiments, the embodiments of the methods of astrocyte differentiation involves performing an assessment at various time points.

In some embodiments, the methods are more readily adapted to scaled-up astrocyte production. In some embodiments of the methods, the astrocyte precursors (e.g., iPSCs and/or NSCs) can be frozen for long-term storage and future generation of astrocytes.

In some embodiments, the scaled-up astrocyte production can be utilized for high-throughput drug screening and other applications in which precursors (e.g., iPSCs and/or NSCs) can be readily thawed and utilized for experiments.

An important functional readout of astrocytes is the support of neuronal synchronous burst activity. The embodiments of the methods disclosed herein generate astrocytes that are functionally identical to primary human astrocytes by microelectrode array (MEA) analysis.

In some embodiments, the present invention is significantly advantageous because the method to derive astrocytes is a serum-free fully defined protocol with growth factors. In some embodiments, methods for producing astrocytes from either pluripotent stem cells and/or from NSCs are described herein. In some embodiments, methods for producing astrocytes from pluripotent stem cells are described herein. In some embodiments, methods for producing astrocytes from NSCs are described herein. In some embodiments, the present invention uses several growth factors and cytokines (FGF, CNTF, LIF, hEBGF). In some embodiments, the several growth factors and cytokines (FGF, CNTF, LIF, hEBGF) are combined in the methods disclosed herein. In some embodiments, the several growth factors and cytokines (FGF, CNTF, LIF, hEBGF) are included earlier in the differentiation to drive glial specification. In some embodiments, the methods employ the use of a base media that mimics CSF osmolarity, which promotes astrocyte differentiation and maintenance. In some embodiments, hEBGF is included as a growth factor that maintains adult astrocytes in a quiescent state.

A major advancement of the embodiments of the methods herein is the generation of astrocytes via co-differentiation with neurons. Incorporation of 3D systems to provide cues for astrocyte development has been previously described. In contrast, in some embodiments, the methods described herein incorporate neuronal cues in 2D to facilitate astrocyte differentiation. The prior art has not attempted to try to establish this in a 2D system. Significant advantages of the 2D culture described herein include ability of undertake large-scale production and high reproducibility of the large-scale production.

Non-limiting applications of the astrocytes generated herein include studying the biology of astrocytes, correlation between astrocyte enriched genes and human health and disease, cell-based therapeutic applications in neurological disease.

In some embodiments, the methods for astrocyte differentiation can be incorporated into a commercial kit for astrocyte generation. In some embodiments, the kit can be utilized to generate astrocytes from patient-derived pluripotent stem cells. In some embodiments, the astrocytes generated from patient-derived autologous pluripotent stem cells can be used for transplantation. In some embodiments, the astrocytes generated from patient-derived autologous pluripotent stem cells can be used for transplantation to prevent and/or treat patients suffering from diseases associated with loss of astrocyte function. Non-limiting examples of diseases include Huntington's disease (HD), Parkinson's disease (PD), Alzheimer's disease (AD), and amyotrophic lateral sclerosis (ALS).

In some embodiments, the astrocytes can be used for treating a neuronal disease or disorder, or for providing defined neuronal cues (non-limiting examples of which include signaling molecules to replace neurons and inclusion of TGFβ to promote the gliogenic switch) and/or to enhance synaptic connections and/or for providing defined neuronal cues in a subject in need thereof, comprising administering a cell, or population of cells or a composition containing the cells or population to the subject. The subject can be a mammal such as a human patient.

### EXAMPLES

The following examples are non-limiting and additional variants are contemplated and within the scope of the disclosure.

### Example 1—Differentiation of iPSCs to Neural Stem Cells (NSCs)

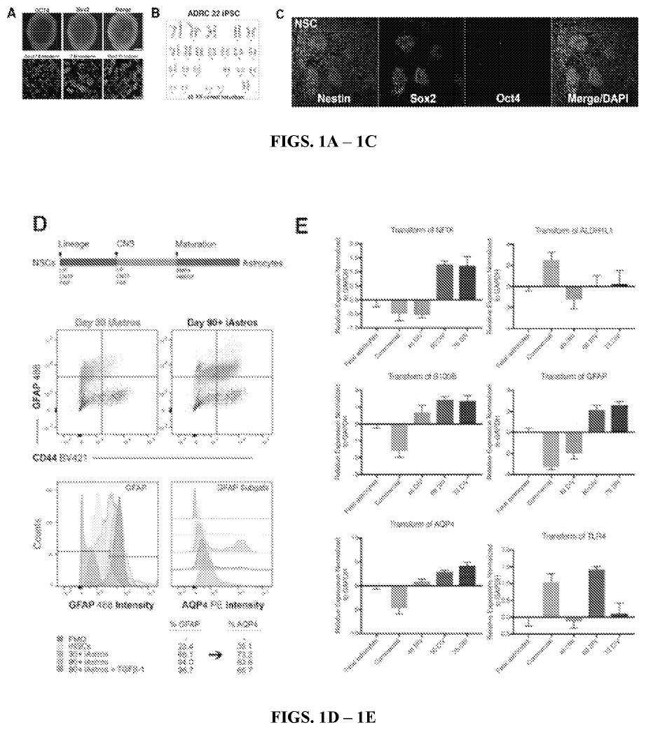

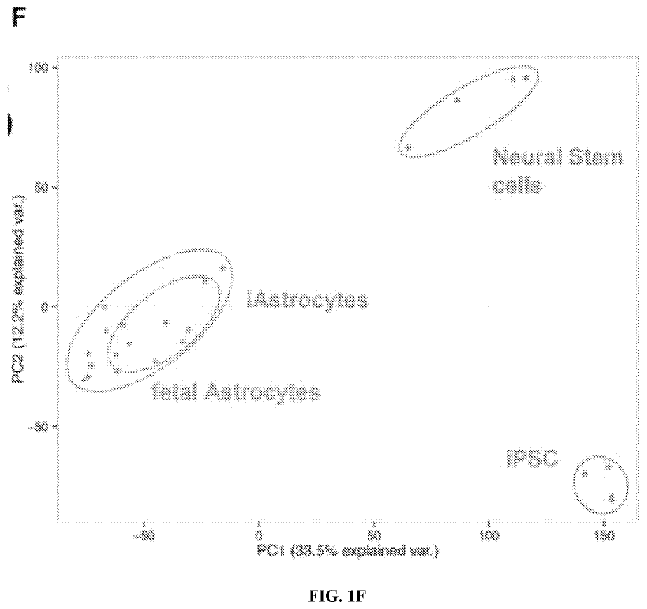

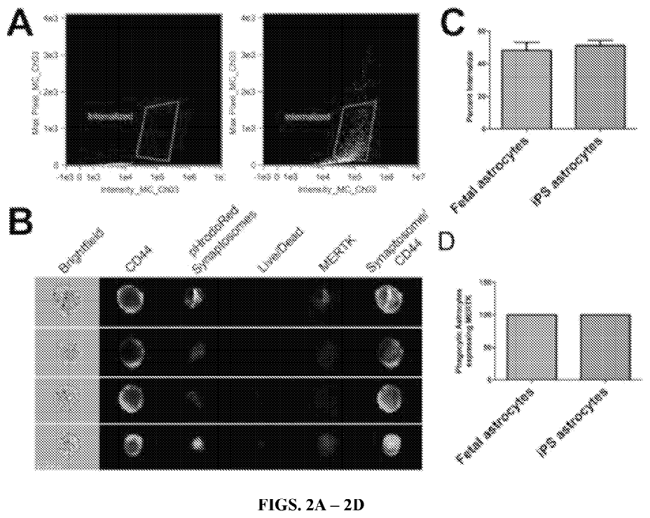

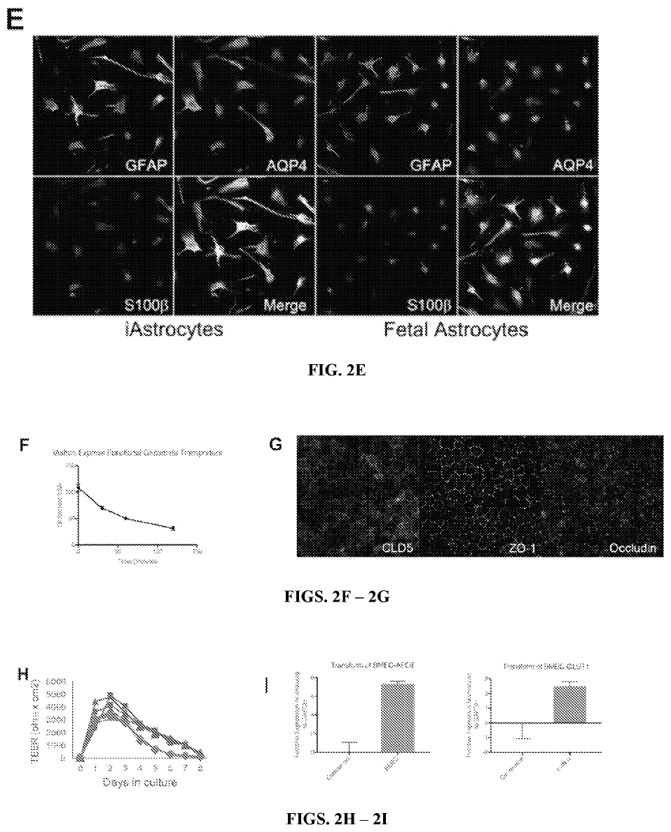

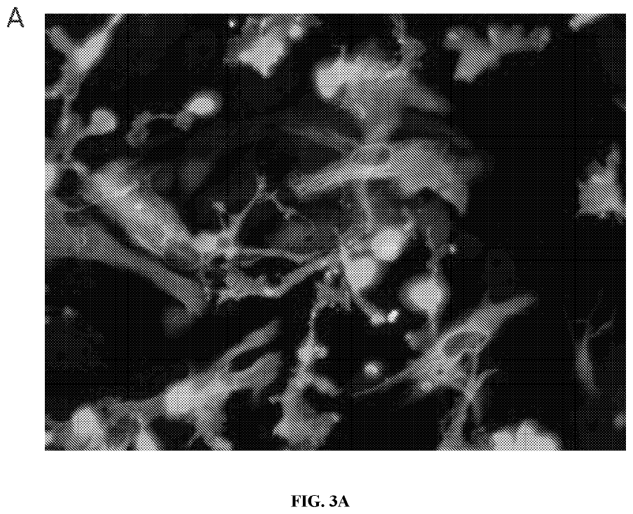

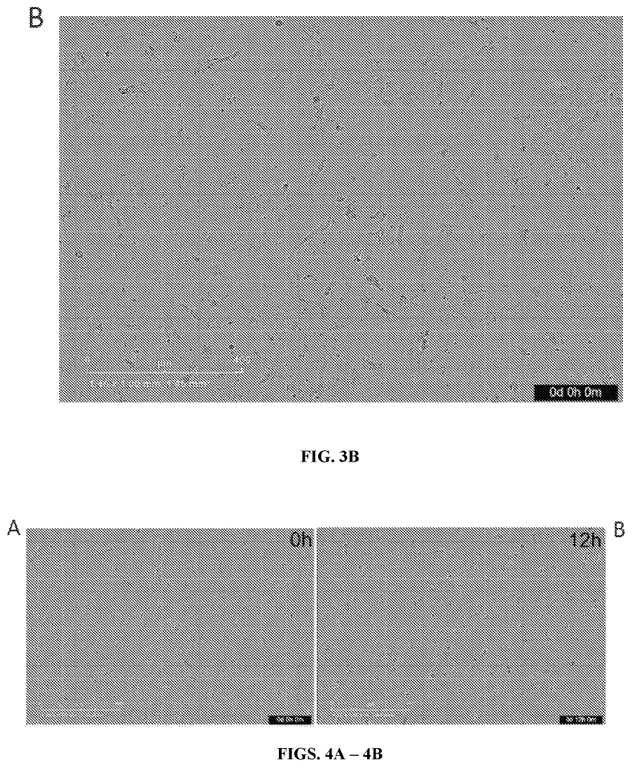

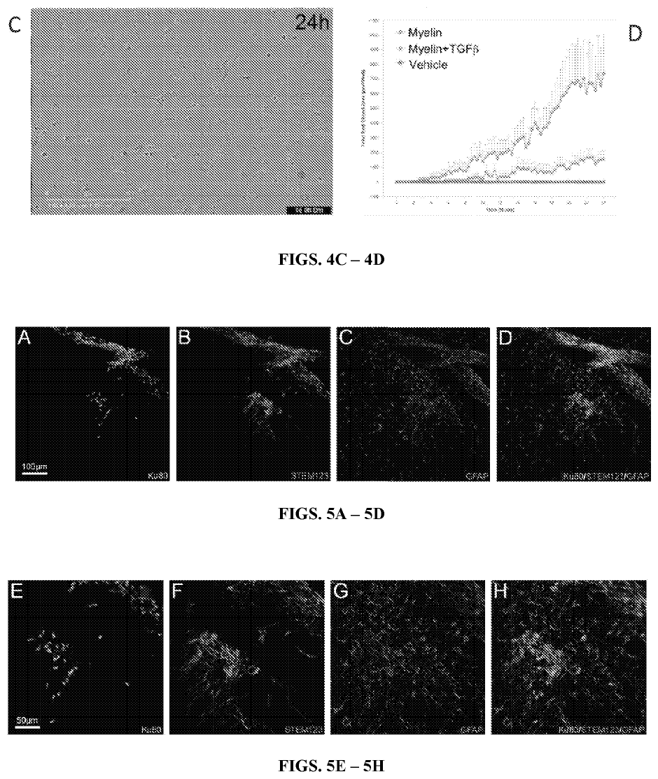

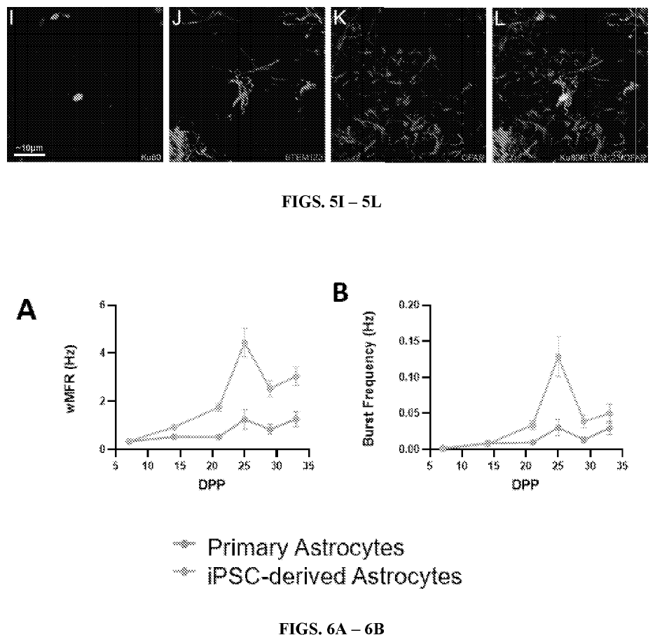

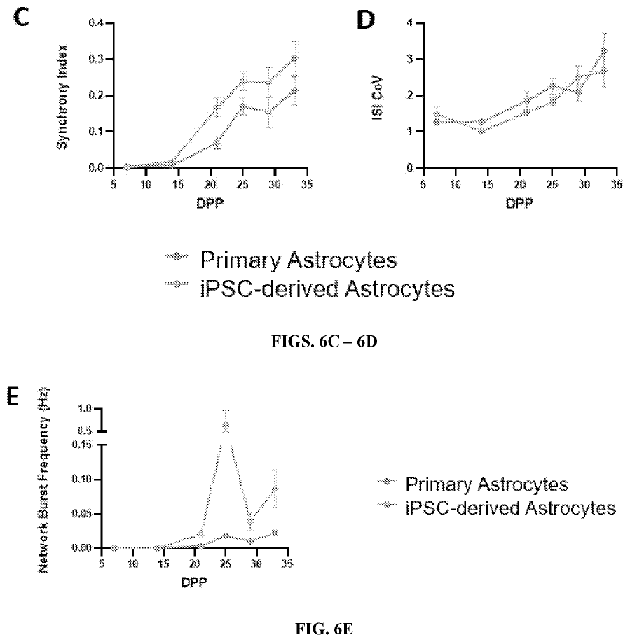

Induced pluripotent stem cells (iPSCs) are differentiated to neural stem cells (NSCs). The protocol is as follows: iPSCs are cultured in feeder-free conditions using completeTeSR-E8 medium and passaged as small colonies at 15-20% confluence. On day 1 of splitting, the medium is replaced with GIBCO Neural Induction Medium, followed by complete media changes every other day. Non-neuronal differentiated cells are manually removed. On day 7, NSCs are harvested using Accutase and expanded on poly-O-laminin-coated plates with Neural Expansion Medium (Gibco). The generation of NSCs is confirmed immunohistochemically by expression of NESTIN and SOX2, markers of NSCs (FIG. 1).

### Example 2—Differentiation of NSCs to Astrocytes

Near confluent NSCs cultured on Matrigel-coated tissue culture (TC) plastic (80-90%) are supplemented with ADM1 such that that ratio of NSC expansion media to ADM1 is 1:1. A 50% media change with ADM1 is performed every 2 days. Cells are passaged at 1:3 onto Matrigel-coated TC plastic on day 7. Briefly, cells are rinsed with pre-warmed HBSS (without Mg2+ and Ca2+)(1×) 3 times and then incubated with pre-warmed Accutase (about 37° C., 5 minutes). Plates containing NSCs are tapped lightly to gently dislodge cells and ADM1 is added to the Accutase at a 2:1 ratio and cells collected by centrifugation (300×g, 5 minutes, at room temperature). A 50% media change is performed every 2 days. On day 15, cells are passage (1:3) onto new Matrigel-coated TC in ADM1:ADM2a/b (1:1). At 30 divisions DIV, cells are passaged (1:3) onto new Matrigel-coated TC plastic in complete Barres media. After 3-4 days, astrocytes are passaged (1:2) to remove neurons onto Matrigel-coated TC plastic in Barres media and further matured.

### Keywords

Astrocyte, astroglia, brain, pluripotent, stem cell, neurodegeneration, spinal cord, iPS, iPSC, iPS-astrocyte, synaptogenesis, synaptic pruning, MEA

### Equivalents

Although the foregoing invention has been described in terms of certain preferred embodiments, other embodiments will be apparent to those of ordinary skill in the art. Additionally, other combinations, omissions, substitutions, and modification will be apparent to the skilled artisan, in view of the disclosure herein. Accordingly, the present invention is not intended to be limited by the recitation of the preferred embodiments but is instead to be defined by reference to the appended claims. All references cited herein are incorporated by reference in their entirety.

The terminology used in the description presented herein is not intended to be interpreted in any limited or restrictive manner and unless otherwise indicated refers to the ordinary meaning as would be understood by one of ordinary skill in the art in view of the specification. Furthermore, embodiments may comprise, consist of, consist essentially of, several novel features, no single one of which is solely responsible for its desirable attributes or is believed to be essential to practicing the embodiments herein described. As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain embodiments and examples, those of ordinary skill in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of ordinary skill in the art based upon this disclosure. It is also contemplated that various combinations or sub-combinations of the specific features and aspects of the embodiments may be made and still fall within the scope of the disclosure. It can be understood that various features and aspects of the disclosed embodiments can be combined with, or substituted for, one another to form varying modes or embodiments of the disclosure. Thus, it is intended that the scope of the present disclosure herein disclosed should not be limited by the particular disclosed embodiments described above.

1. A method of producing neural stem cells (NSCs) from human induced progenitor cells (iPSCs), comprising culturing the iPSCs in completeTeSR-E8 culture medium in the absence of feeder cells for an effective amount of time, thereby producing human neural stem cells from human iPSCs.

2. The method of claim 1, wherein the culturing comprises passaging iPSCs as small colonies at 15-20% confluence from day 0 to day 1.

3. The method of claim 2, further comprising replacing the culture medium on day 1 with GIBCO Neural Induction Medium in the absence of feeder cells.

4. The method of claim 3, further comprising complete media changes on days 3 and day 5 with GIBCO Neural Induction Medium in the absence of feeder cells, optionally further comprising removing non-neuronal differentiated cells.

5. (canceled)

6. The method of claim 4, further comprising, harvesting NSCs using Accutase on day 7 and expanding on poly-O-laminin-coated plates with Neural Expansion Medium (NSC expansion medium) in the absence of feeder cells.

7. The method of claim 6, comprising confirming the generation of NSCs by immunohistochemistry for expression of markers of NSCs, optionally wherein the markers of NSCs comprise NESTIN and SOX2.

8. (canceled)

9. A method of producing human astrocytes from human neural stem cells, comprising culturing NSCs obtained by the method of claim 6 in NSC in culture media comprising expansion medium on Matrigel-coated tissue culture (TC) plastic to a confluence of about 80-90%.

10. The method of claim 9, further comprising supplementing the culture medium with ADM1 such that the a ratio of NSC expansion media to ADM1 is 1:1.

11. The method of claim 10, further comprising performing a 50% media change with ADM1 every 2 days.

12. The method of claim 11, further comprising passaging cells at 1:3 onto Matrigel-coated TC plastic on day 7, optionally wherein the passaging comprises:
rinsing the cells with pre-warmed 1× HBSS (without Mg2+ and Ca2+) 3 times;
incubating with pre-warmed Accutase at 37° C. for 5 minutes;
tapping plates containing the cells dislodge the cells;
adding ADM1 to the Accutase at a 2:1 ratio; and
collecting the cells by centrifugation at 300×g for 5 minutes at room temperature.

13. (canceled)

14. The method of claim 12, further comprising performing a 50% media change every 2 days.

15. The method of claim 14, further comprising passaging one-third of the cells on day 15 onto new Matrigel-coated TC plastic in ADM1:ADM2a/b at a 1:1 ratio.

16. The method of claim 15, further comprising passaging one-third of the cells at 30 divisions onto new Matrigel-coated TC plastic in complete Barres media.

17. The method of claim 16, further comprising passaging after 3-4 days one-half of the astrocytes generated to remove neurons onto Matrigel-coated TC plastic in Barres media.

18. The method of claim 17, further comprising further maturing the astrocytes.

19. An isolated human astrocyte prepared by the method of claim 9.

20. A population of isolated human astrocytes of claim 19.

21. A composition comprising the isolated human astrocyte of claim 19 and a carrier.

22. A method to deliver human astrocytes to a tissue, comprising contacting the tissue with the isolated human astrocyte of claim 19.

23. A method to treat a neurological disease or disorder in a subject in need thereof, comprising administering the isolated human astrocyte of claim 19.
